(19) United States
(12) Patent Application Publication
(10) Pub. No.: US 2022/0085628 A1
(43) Pub. Date: Mar. 17, 2022

(54) ELECTRICAL ISOLATION DURING BATTERY CHARGING OF WEARABLE DEVICES

(71) Applicant: Soovu Labs, Inc., Seattle, WA (US)

(72) Inventor: Allan H. Stephan, Seattle, WA (US)

(21) Appl. No.: 17/309,894

(22) PCT Filed: Dec. 24, 2019
(86) PCT No.: PCT/US2019/068477

§ 371 Date: Jun. 28, 2021

Related U.S. Application Data
(60) Provisional application No. 62/785,526, filed on Dec. 27, 2018.

(51) Int. Cl. H02J 7/00; A61F 7/02; H02J 50/00

(52) U.S. Cl. CPC H02J 7/0044; A61F 7/02; A61H 2201/0207; A61H 2201/165; H02J 50/005

(57) ABSTRACT

A modular stimulus applicator system and method are disclosed. The system includes a plurality of wirelessly controlled stimulus pods configured to be anchored to a patients body, and configured to deliver stimulus to the patients body. The stimulus can be heat, vibration, or electrical stimulus, or any combination thereof. The stimulus pods can be charged by a charging station that prevents the stimulus pods from being in contact with the patients body during charging.

### TECHNICAL FIELD

The following disclosure relates generally to stimulus-based therapeutic devices, systems, and methods. In particular, the disclosure relates to systems and methods for applying heat, vibration, electrical, and other stimulus to a patient's body for therapeutic purposes.

### BACKGROUND

In 1965, Melzack and Wall described the physiologic mechanisms by which stimulation of large diameter non-pain sensory nerves could reduce the amount of unpleasant activity carried by pain nerves. This landmark observation published in Science was termed the “gate control theory” and offered a model to describe the interactions between various types of the sensory pathways in the peripheral and central nervous systems. The model described how non-painful sensory input such as mild electrical stimulation could reduce or gate the amount of nociceptive (painful) input that reached the central nervous system.

The gate-control theory stimulated research that lead to the creation of new medical devices such as transcutaneous electrical nerve stimulators (TENS). In brief, TENS works by electrically “blocking” pain impulses carried by peripheral nerves. Receptors to cold and heat are located just below the surface of the skin. Heat receptors are activated through a temperature range of about 36° C. to 45° C. and cold receptors by a temperature range about 1-20° C. below the normal skin temperature of 34° C. (Van Hees and Gybels, 1981). The stimuli are transmitted centrally by thin poly-modal C nerve fibers. Activation of heat receptors are also affected by the rate of rise of the heat stimuli (Yarnitsky, et al., 1992). Above 45° C. warm receptor discharge decreases and nociceptive response increases producing the sensations of pain and burning (Torebjork et al., 1984).

Activation of poly-modal thermal receptors causes significant pain relief in controlled experimental conditions. Kakigi and Watanabe (1996) demonstrated that warming and cooling of the skin in human volunteers could significantly reduce the amount of reported pain and somatosensory evoked potential activity induced by the noxious stimulation of a CO2 laser. The authors offered that the effects seen could be from a central inhibitory effect produced by the thermal stimulation. Similar inhibition of pain from thermal simulation was reported in a different Human experimental pain model (Ward et al., 1996). The study authors (Kakigi and Watanabe 1996 and Ward et al., 1996) proposed that the thermal analgesia was in part from a central inhibitory effect (gating) from stimulation of small thin C nerve fibers. This contrasts with TENS which produces at least part of its analgesia through gating brought on by activation of large diameter afferent nerve fibers.

A number of recent clinical studies strongly support the use of heat as an analgesic in patients who suffer from chronic pain and offer potential mechanisms by which heat produces analgesia. Abeln et al. (2000) in a randomized controlled single-blinded study examined the effect of low level topical heat in 76 subjects who suffered from low back pain. Heat treatment was statistically more effective in relieving pain and improving the quality of sleep than that produced by placebo.

Weingand et al. (2001) examined the effects in a randomized, single blinded, controlled trial of low level topical heat in a group of over 200 subjects who suffered from low back pain and compared heat to placebo heat, an oral analgesic placebo, and ibuprofen 1200 mg/day. The authors found heat treatment more effective than placebo and superior to ibuprofen treatment in relieving pain and increasing physical function as assessed by physical examination and the Roland Morris disability scale.

A separate group (Nadler at al., 2002) found similar results in a prospective single blinded randomized controlled trial of 371 subjects who suffered from acute low back pain. The authors found that cutaneous heat treatment was more effective than oral ibuprofen 1200 mg/day, acetaminophen 4000 mg/day or oral and heat placebos in producing pain relief and improving physical function. The authors offered several hypotheses for the mechanism(s) of action which includes increased muscle relaxation, connective tissue elasticity, blood flow, and tissue healing potential provided through the low-level topical heat. Similar beneficial effects of topical heat were show in patients who suffered from dysmenorrhea (Akin et al., 2001), and temporomandibular joint pain TMJ (Nelson et al., 1988).

A recent study used power Doppler ultrasound to evaluate the effects of topical heat on muscle blood flow in Humans (Erasala et al., 2001). Subjects underwent 30 minutes of heating over their trapezius muscle and changes in blood flow were examined at 18 different locations over the muscle. Vascularity increased 27% (p=0.25), 77% (p=0.03) and 104% (p=0.01) with 39° C., 40° C., or 42° C. temperature of the heating pad. Importantly increases in blood flow extended approximately 3 cm deep into the muscle. The authors concluded that the increased blood flow likely contributed to the analgesic and muscle relaxation properties of the topical heat. Similar increases in deep vascular blood flow were noted using magnetic resonance thermometry in subjects treated with mild topical heat by two separate groups (Mulkern et al., 1999, and Reid et al., 1999).

Recent studies demonstrated the analgesic effectiveness of heat and provided potential mechanisms of action. The mechanisms include a reduction of pain through a central nervous system interaction mediated via thin c-fibers (Kakigi and Watanabe, 1996, Ward et al. 1996), enhancement of superficial and deeper level blood flow (Erasala et al., 2001, Mulkern et al., 1999, Reid et al., 1999), or local effects on the muscle and connective tissue (Nadler et al., 2002, Akin et al. 2001). TENS is thought to act through inhibition of nociception by increasing endogenous opioids or by a neural inhibitory interaction of nociception via large diameter fibers. It is likely that TENS and heat act partly through different mechanisms with the potential for enhanced or even synergistic interactions. TENS is widely used and endorsed by the pain management guidelines of both the AHCPR and American Geriatric Society (Gloth 2001). However, a significant number of patients fail to achieve adequate relief with TENS or fail within six months of starting treatment (Fishbain et al., 1996).

### BRIEF DESCRIPTION OF THE DRAWINGS

Many aspects of the present technology can be better understood with reference to the following drawings. The components in the drawings are not necessarily to scale. Instead, emphasis is placed on clearly illustrating the principles of the present technology.

FIG. 1A is a perspective view of a stimulus pod system configured in accordance with an embodiment of the present technology.

FIG. 1B is an exploded view of a stimulus pod of the system shown in FIG. 1A configured in accordance with an embodiment of the present technology.

FIG. 1C is an exploded view of an anchor of the system shown in FIG. 1A configured in accordance with an embodiment of the present technology.

FIGS. 2A-2C are side cross-sectional views of the system shown in FIG. 1A illustrating a stimulus pod secured against an anchor in accordance with embodiments of the present technology.

FIGS. 3A-3C are enlarged side-cross sectional views of a portion of the system shown in FIG. 2A and illustrating various attachment mechanisms for attaching the stimulus pod to the anchor in accordance with embodiments of the present technology.

FIGS. 3D and 3E are enlarged side-cross sectional views of a portion of the system shown in FIG. 2B and illustrating various attachment mechanisms for attaching the stimulus pod to the anchor in accordance with embodiments of the present technology.

FIG. 3F is a bottom cross-sectional view of the system shown in FIG. 2C and illustrating an attachment mechanism for attaching the stimulus pod to the anchor in accordance with an embodiment of the present technology.

FIG. 4 is a perspective view of a stimulus pod configured in accordance with an embodiment of the present technology.

FIG. 5 is a partially schematic view of a stimulus delivery system configured in accordance with an embodiment of the present technology.

FIG. 6 is a partially schematic view of a stimulus delivery system configured in accordance with another embodiment of the present technology.

FIG. 7A is a perspective view of a charging station configured in accordance with an embodiment of the present technology.

FIG. 7B is an enlarged view of a portion of the charging station shown in FIG. 7A.

FIGS. 8A and 8B are a bottom-perspective view and a side view, respectively, of a stimulus pod configured in accordance with embodiments of the present technology.

FIGS. 9A-90 are a front-perspective view, a rear-perspective view, and a side cross-sectional view, respectively, of the stimulus pod of FIGS. 8A and 8B positioned on the charging station of FIGS. 7A and 7B in accordance with embodiments of the present technology.

FIG. 10A is a perspective view of a charging station configured in accordance with another embodiment of the present technology.

FIG. 10B is an enlarged, side cross-sectional view of a portion of the charging station shown in FIG. 10A.

FIGS. 10C and 10D are perspective views of a stimulus pod positioned above and positioned on, respectively, the charging station shown in FIGS. 10A and 10B in accordance with embodiments of the present technology.

FIG. 11A is an illustration of a mapping of thermal receptors of a human leg and foot.

FIG. 11B is a graph of the excitation of thermal receptors versus applied heat.

FIG. 12 is a back view of a human form with a plurality of attached stimulus pods configured in accordance an embodiment of the present technology.

FIG. 13 is a graph of temperature versus time illustrating a variable heat cycle in accordance with an embodiment of the present technology.

FIG. 14 is a graph of temperature versus time illustrating a variable heat cycle in accordance with another embodiment of the present technology.

FIG. 15 is a graph of energy applied versus time illustrating the resultant skin temperature of a patient in accordance with an embodiment of the present technology.

FIG. 16 is a graph of energy applied versus time in accordance with another embodiment of the present technology.

FIG. 17 illustrates energy applied to exemplary thermal zones and the resultant skin temperature of a patient in accordance with an embodiment of the present technology.

FIG. 18 illustrates an on-demand pattern of variable heat cycles over time as requested by a patient in accordance with an embodiment of the present technology.

FIGS. 19A-19C are a top view, a side view, and end view, respectively, of a charging station having stimulus pods positioned thereon in accordance with another embodiment of the present technology.

FIGS. 19E and 19F are top views of the charging station of FIGS. 19A-19C with the stimulus pods removed.

FIGS. 19F and 19G are side and end cross-sectional views, respectively, of the charging station of FIGS. 19A-19C in accordance with an embodiment of the present technology.

FIGS. 19H and 19I are side and end cross-sectional views, respectively, of the charging station of FIGS. 19A-19C in accordance with another embodiment of the present technology.

FIGS. 19J-19M are end views of charging station 19A-19C having a lid configured in accordance with embodiments of the present technology.

FIG. 20 is a side view of a charging station having a stimulus pod positioned therein in accordance with another embodiment of the present technology.

### DETAILED DESCRIPTION

The present technology is directed generally to systems, devices, and associated methods for applying stimuli to various parts of the body of a human subject or patient using a series of modular pods. The pods can be controlled by a remote controller in the form of a computer (e.g., a desktop computer, a laptop computer, etc.), or a mobile device (e.g., a mobile phone, tablet, MP3 player, etc.). The pods can releasably attach to disposable anchors that adhere to the body at various locations to which the patient desires to direct heat therapy.

The present technology is further directed to charging stations for recharging the pods. In several of the embodiments described below, a charging station is configured to mechanically prevent the simultaneous charging of a pod and attachment of the pod to a skin surface of the patient.

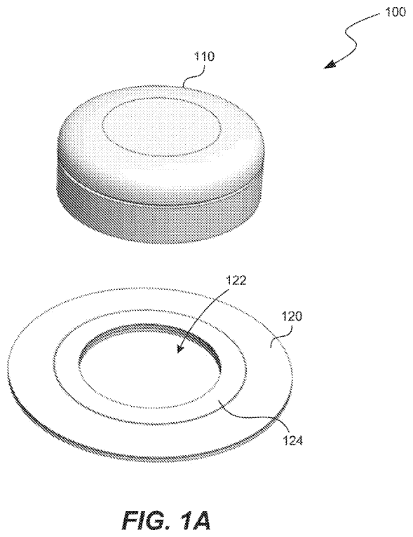

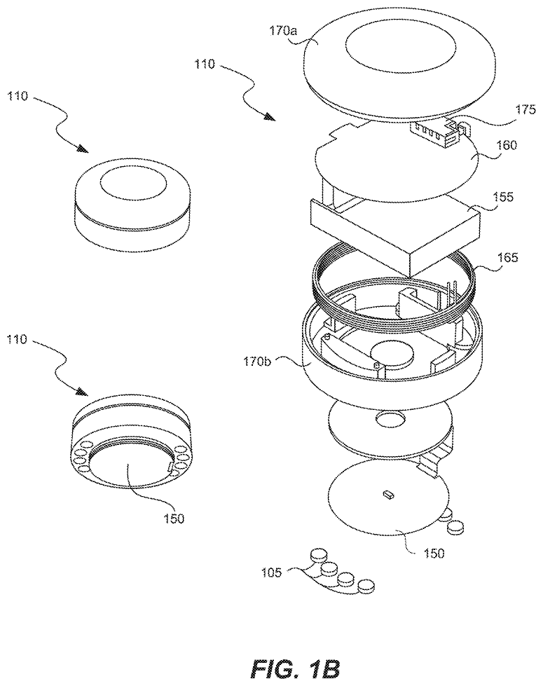

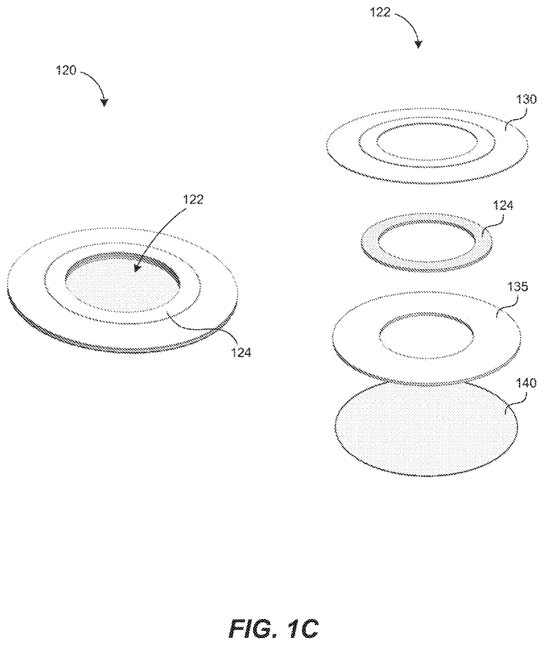

Several details describing thermal and electrical principles are not set forth in the following description to avoid unnecessarily obscuring embodiments of the present technology. Moreover, although the following disclosure sets forth several embodiments of the present technology, other embodiments can have different configurations, arrangements, and/or components than those described herein without departing from the spirit or scope of the present technology. For example, other embodiments may have additional elements, or they may lack one or more of the elements described in detail below with reference to FIGS. 1-18.

The terminology used in the description presented below is intended to be interpreted in its broadest reasonable manner, even though it is being used in conjunction with a detailed description of certain specific embodiments of the present technology. Certain terms may even be emphasized below; however, any terminology intended to be interpreted in any restricted manner will be overtly and specifically defined as such in this Detailed Description section. Additionally, the present technology can include other embodiments that are within the scope of the claims, but are not described in detail with respect to FIGS. 1-18.

Reference throughout this specification to “one embodiment” or “an embodiment” means that a particular feature, structure or characteristic described in connection with the embodiment is included in at least one embodiment of the present technology. Thus, the appearances of the phrases “in one embodiment” or “in an embodiment” in various places throughout this specification are not necessarily all referring to the same embodiment. Furthermore, the particular features or characteristics may be combined in any suitable manner in one or more embodiments.

The terms used herein are not intended—and should not be taken—to exclude from the scope of this present technology other types of heat sources that are designed to be placed on the skin to enable pain relief. Illustrative embodiments will be shown and described; however, one skilled in the art will recognize that the illustrative embodiments do not exclude other embodiments.

The headings provided herein are for convenience only and do not interpret the scope or meaning of the claimed present technology.

### I. Selected Embodiments of Stimulus Pod Systems

FIG. 1A is a perspective view of a stimulus pod system 100 (“system 100”) configured in accordance with an embodiment of the present technology. In the illustrated embodiment, the system 100 includes a stimulus pod 110 and an anchor 120. The stimulus pod 110 can be between about 0.5 inch to 2 inches in diameter (e.g., about 1 inch in diameter) and can be equipped to deliver different stimuli to a patient's body, including heat, vibration, and/or electricity. In some embodiments, the stimulus pod 110 can include sensors that gather information and relay the information back to a control station. The anchor 120 can have an adhesive surface that can be applied to various locations on the patient's body, an aperture 122, and an attachment ring 124 that can engage the stimulus pod 110 to hold the stimulus pod 110 onto the patient's body. Additionally or alternatively, the stimulus pod 110 can be kept in place by clothing, magnets, a Velcro-type applicator, elastic bands, pocket-like holders, braces, or other type of applicators capable of holding the stimulus pod 110 against the patient's skin.

FIG. 1B is an exploded view of the pod 110 shown in FIG. 1A configured in accordance with an embodiment of the present technology. In the illustrated embodiment, the stimulus pod 110 includes a stimulus surface 150 that contacts the patient's skin to deliver heat, mild electrical stimuli, vibration, and/or other stimuli to the patient's body in a measured, deliberate pattern to relieve pain and discomfort in the patient's body. In the illustrated embodiment, the stimulus pod 110 includes a battery 155, a circuit board 160, a charging coil 165, and several housing elements 170 (individually referred to as an upper cover 170a and a body 170b). The battery 155 can power the stimulus surface and the circuit board 160. The battery 155 can be a lithium polymer battery or another suitable battery type. The charging coil 165 can be configured to receive power from a power source (e.g., a charging station) and deliver the power to the battery 155. In some embodiments, the stimulus pod 110 can include a wireless communication link 175 through which the stimulus pod 110 receives instructions and/or sends data to and from a control station. The housing elements 170 can enclose the internal components of the stimulus pod 110 and provide a convenient handling surface.

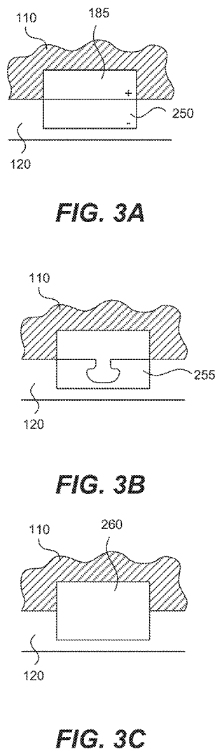

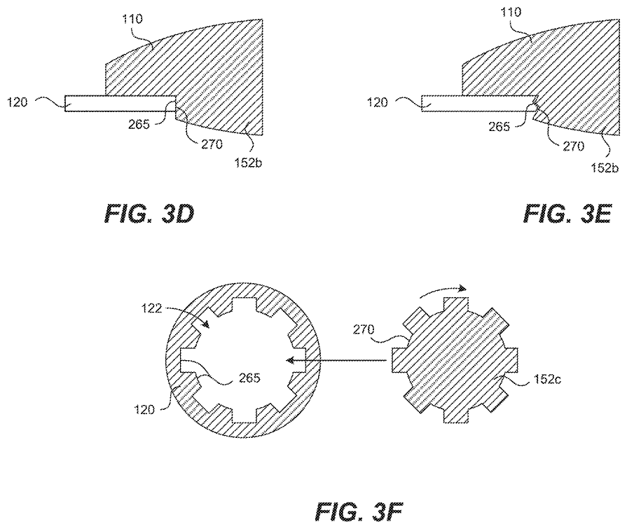

In some embodiments, as described in greater detail below with reference to FIGS. 3A-3F, the stimulus pod 110 can include attachment means to attach the stimulus pod 110 to the anchor 120. Referring to FIGS. 1A and 1B, for example, the stimulus pod 110 can have metal slugs 105 that can be magnetized and coupled to the attachment ring 124 (e.g., a metallic ring) in the anchor 120 to hold the stimulus pod 110 to the anchor 120. In some embodiments, the metal slugs 105 can also be used for stimulus delivery. In some embodiments, the metal slugs 105 can be positioned on a top side of the stimulus pods 110 and can be used to interface with a charging station and/or other external device.

In some embodiments, multiple ones of the stimulus pods 110 can be used in concert at different places on the patient's body. In some embodiments, the stimulus pods 110 can also be used to deliver medicine to a patient through electrophoresis, iontophoresis, and/or heat-enhanced perfusion due to capillary dilation. Electrophoresis is the motion of dispersed particles relative to a fluid under the influence of a spatially uniform electric field. Electrophoresis is ultimately caused by the presence of a charged interface between the particle surface and the surrounding fluid. Iontophoresis (a.k.a., Electromotive Drug Administration (EMDA)) is a technique using a small electric charge to deliver a medicine or other chemical through the skin. It is basically an injection without the needle. The technical description of this process is a non-invasive method of propelling high concentrations of a charged substance, normally a medication or bioactive agent, transdermally by repulsive electromotive force using a small electrical charge applied to an iontophoretic chamber containing a similarly charged active agent and its vehicle. One or two chambers are filled with a solution containing an active ingredient and its solvent, also called the vehicle. The positively charged chamber (anode) will repel a positively charged chemical, whereas the negatively charged chamber (cathode) will repel a negatively charged chemical into the skin.

FIG. 1C is an exploded view of the anchor shown in FIG. 1A configured in accordance with an embodiment of the present technology. In the illustrated embodiment, the anchor 120 includes the attachment ring 124, an upper surface 130, an adhesive layer 135, and a liner 140. In some embodiments, the liner 140 can be removed to expose the adhesive layer 135 before placing the anchor 120 on the patient's body. The upper surface 130 is exposed to ambient conditions and accordingly can be similar to a bandage or a wound covering to provide a clean, water-resistant surface for the anchor 120. The attachment ring 124 is positioned beneath the upper surface 130 and can be a metallic ring, such as a steel ring, that can be coupled to the magnetic metal slugs 105 of the stimulus pod 110 and/or other components of the stimulus pod 110. The attachment ring 124 is held to the upper surface 130 by the adhesive layer 135, which can have an adhesive on (i) an upper side thereof to adhere to the attachment ring 124 and the upper surface 130 and (ii) on a lower side thereof to adhere to the liner 140. The materials used to form the anchor 120 can all be rigid enough to maintain a proper shape, but flexible enough to substantially conform to the patient's body. For example, the attachment ring 124 can be segmented or thin to permit the anchor 120 to flex to some degree.

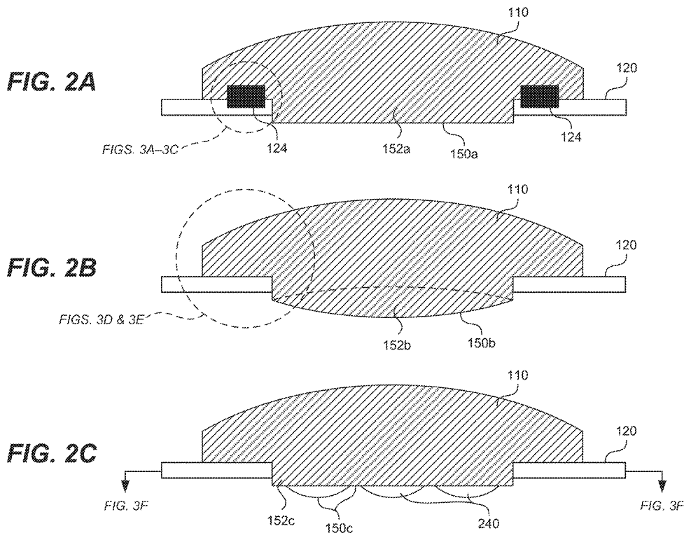

Referring to FIGS. 1A-1C together, once the anchor 120 is placed on the body of the patient (e.g., adhered to the body of the patient via the adhesive layer 135), the stimulus pod 110 can be placed into the aperture 122 in the anchor 120 and held in contact with the patient's body to deliver heat and/or other stimulation to the patient. Specifically, in many applications, the stimulus from the stimulus pod 110 can be delivered to the patient's body with the stimulus surface 150 directly contacting the patient's skin. Accordingly, at least a portion of the stimulus pod 110 can project beyond/through the anchor 120 such that the stimulus surface is in direct contact with the patient's skin. FIGS. 2A-2C, for example, are side cross-sectional views of the system 100 with the stimulus pod 110 secured against the anchor 120 and having various stimulus surfaces 150 (individually labeled as stimulus surfaces 150a-150c) in accordance with embodiments of the present technology.

Referring to FIG. 2A, in some embodiments the stimulus pod 110 can have a plug 152a that extends slightly beyond (e.g., projects past a lower surface of) the anchor 120. In the illustrated embodiment, the plug 152a has a stimulus surface 150a with a flat (e.g., generally planar) profile. The attachment ring 124 can engage the stimulus pod 110 with sufficient force such that the stimulus surface 150a presses down onto the patient's skin to ensure sufficient contact with the skin.

Referring to FIG. 2B, in some embodiments the stimulus pod 110 can have a plug 152b with a convex stimulus surface 150b that extends beyond the anchor 120. The slope of the convex stimulus surface 150b can depend in part on the size of the stimulus pod 110 and its intended application. For example, the slope can be selected such that substantially the entire stimulus surface 150b contacts the patient's skin (e.g., such that the slope of the stimulus surface 150b is not too extreme). Accordingly, the convex stimulus surface 150b can have relatively more surface area than the flat stimulus surface 150a shown in FIG. 2A. Therefore, in some embodiments the stimulus surface 150b can contact a relatively greater area of the skin of the patient than the flat stimulus surface 150a.

Referring to FIG. 2C, in some embodiments the stimulus pod 110 can have a plug 152c that extends beyond the anchor 120 and that has a stimulus surface 150c with several small bumps or projections 240. The dimensions of the stimulus surface 150c and the projections 240 can be selected to increase the surface area of the stimulus surface 150c that contacts the patient's skin without creating void spaces or air pockets between the projections 240 that might reduce effective heat transfer or delivery of other stimuli or drugs. In some embodiments, the projections 240 are not discrete, but are continuous and/or sinusoidal.

The stimulus pod 110 can be attached to (e.g., secured against, retained by, etc.) the anchor 120 such that the stimulus surface 150 is secured against the patient's skin. FIGS. 3A-3F, for example, illustrate various attachment mechanisms for attaching the stimulus pod 110 to the anchor 120 in accordance with embodiments of the present technology. More specifically, FIGS. 3A-3C are enlarged side-cross sectional views of a portion of the system 100 shown in FIG. 2A, FIGS. 3D and 3E are enlarged side-cross sectional views of a portion of the system 100 shown in FIG. 2B, and FIG. 3F is a bottom cross-sectional view of the system 100 shown in FIG. 2C taken along the line shown in FIG. 2C.

Referring to FIG. 3A, in some embodiments the anchor 120 can include a metallic or magnetic ring 250 that corresponds to a magnet 185 in the stimulus pod 110. The magnetic force between the ring 250 and the magnet 185 can hold/secure the stimulus pod 110 in place relative to the anchor 120.

Referring to FIG. 3B, in some embodiments the anchor 120 can be held/secured to the stimulus pod 110 by a mechanical fastener 255 such as a snap, or other similar mechanical attachment means. In the illustrated embodiment, the anchor 120 includes a resilient recession and the stimulus pod 110 includes a matching, resilient projection that, when pressed together, mechanically hold the stimulus pod 110 in place on the anchor 120b. In other embodiments, the anchor 120 can include a projection and the stimulus pod 110 can include a mating recession. In some embodiments, the attachment mechanism at the interface between the anchor 120 and the stimulus pod 110 can operate along the same principle as a plastic cap on a cardboard cup, such as a coffee cup and lid.

Referring to FIG. 3C, in some embodiments the anchor 120 can be held/secured to the stimulus pod 110 by a hook-and-loop fastener 260.

Referring to FIG. 3D, in some embodiments the anchor 120 can include an interior surface 265 that engages an exterior surface 270 of the plug 152b of the stimulus pod 110 to secure the stimulus pod 110 to the anchor 120. More particularly, one or both of the surfaces 265, 270 can be formed of a resilient material such that, when the plug 152b is pressed into the aperture 122 in the anchor 120, the plug 152b snaps into place.

Referring to FIG. 3E, in some embodiments the surfaces 265, 270 can have corresponding/mating threads such that the stimulus pod 110 can be screwed into the anchor 120.

Referring to FIG. 3F, in some embodiments the interior surface 265 of the anchor can have a keyed, regular, irregular, or other pattern, and the exterior surface 270 of the stimulus pod 110 can include a corresponding/matching pattern configured to engage with the anchor 120 to hold the stimulus pod 110 in place.

Any of the attachment mechanisms illustrated in FIGS. 3A-3F provide a simple way for the patient to apply a stimulus pod 110 to their body. As one of ordinary skill in the art will appreciate, the various configurations of the anchor 120 and stimulus pod 110 shown in FIGS. 2A-3F can be combined and/or integrated together (e.g., to include a magnetic and friction-fit connection).

During operation of the system 100, multiple ones of the stimulus pods 110 can be interchanged between different ones of the anchors 120, and vice versa. A patient can use a stimulus pod 110 until the battery is depleted, and then simply swap in another stimulus pod 110 with a fresh battery. The attachment means can be strong enough and the dimensions of the stimulus pod 110 can be small enough that the stimulus pod 110 can be worn under the patient's clothing easily. The placement of the anchors 120 can vary greatly according to a predetermined diagnostic pattern or personal preference. In some embodiments, one or more of the stimulus pods 110 can be placed at an area of discomfort, such as a painful lower back. Some research suggests that placing additional stimulus pods 110 at an area remote from a problem area can also provide analgesic effects. For example, a patient may place one of the stimulus pods 110 at the lower back—where their pain is—but they can also use a second one of the stimulus pods 110 near the shoulders or on the legs. Multiple stimulus pods 110 can be used in concert to produce an aggregate affect. Because different areas of the human body have different nerve densities, in certain areas two of the stimulus pods 110 placed near one another can be perceived as a single, large stimulus pod. For example, the patient's back has much lower nerve density than the face, neck, or arms. Accordingly, the patient can use a pair of small stimulus pods 110 (e.g., one or two inches in diameter) at the lower back and spaced about three or four inches apart to achieve the same sensory result as a larger stimulus pod covering the entire area. An unexpected benefit of this arrangement is that much less power is required to provide the stimulus in two small areas than would be required to stimulate the entire area.

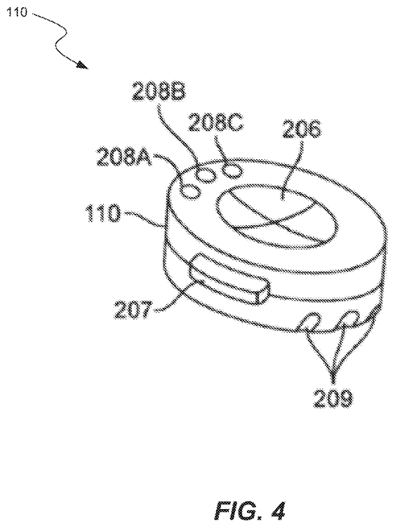

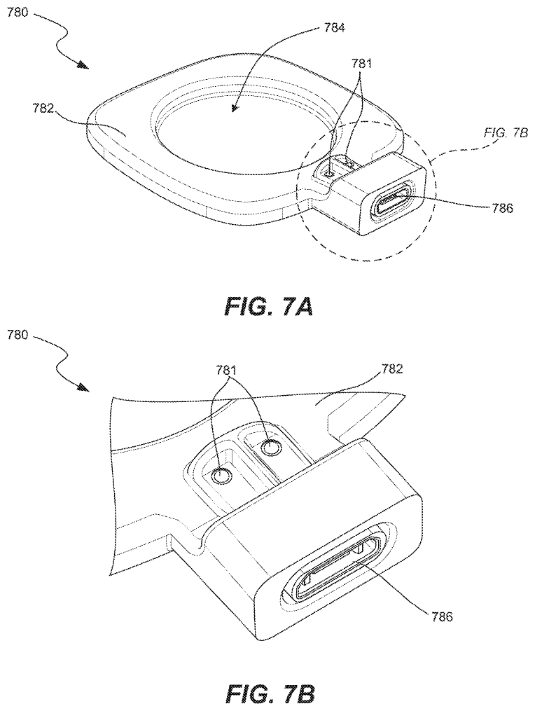

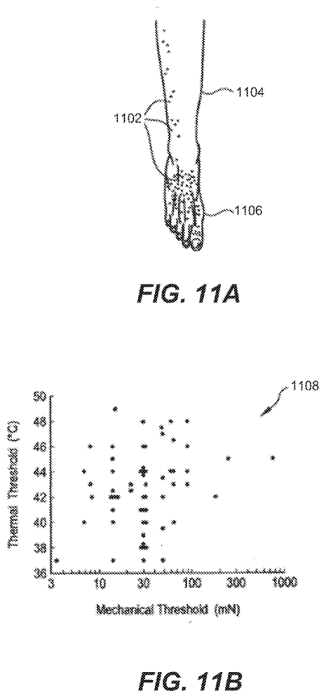

FIG. 4 is a perspective view of the stimulus pod 110 and showing additional features of the stimulus pod 110 in accordance with embodiments of the present technology. In the illustrated embodiment, the stimulus pod 110 has contacts 209 for interfacing with a charging station (e.g., as described in detail below with reference to FIGS. 7A & 7B) and that are positioned on a lower surface of the stimulus pod 110. In other embodiments, the contacts 209 can be positioned on an upper surface of the stimulus pod 110 or elsewhere on the stimulus pod 110. In the illustrated embodiment, the stimulus pod 110 further includes an on/off switch 207 for powering/de-powering the stimulus pod 110. Although a simple push-type on/off switch is illustrated, in other embodiments, the stimulus pod 110 can include other types of switches including, for example, a slide switch, an optical switch, a touch sensor, an accelerometer to detect tapping, etc. In use, the on/off switch 207 is typically activated after contact with the patient's skin has been established. In addition to its power on/off function, the on/off switch 207 can be configured to control a number of heat cycles and/or a temperature of the stimulus pod 110 (e.g., as described in detail below with reference to FIGS. 11A-18).

In the illustrated embodiment, the stimulus pod 110 also includes a stimulus cycle switch 206 configured to, for example, switch between different levels of an applied stimulus (e.g., a low, medium, or high temperature). The stimulus pod 110 can also include indicators 208A-C such as LEDs that can light up in response to a particular setting of the stimulus cycle switch 206. In other embodiments, a single indicator 208 capable of changing its color, intensity, or other property can be used to indicate different settings of the stimulus pod 110. A push-type stimulus cycle switch 206 is illustrated in FIG. 4, however, in other embodiments other types of switches can be used such as, for example, a slide switch, multi-pole throw switch, touch sensitive switch, etc.

### II. Selected Embodiments of Stimulus Delivery Systems

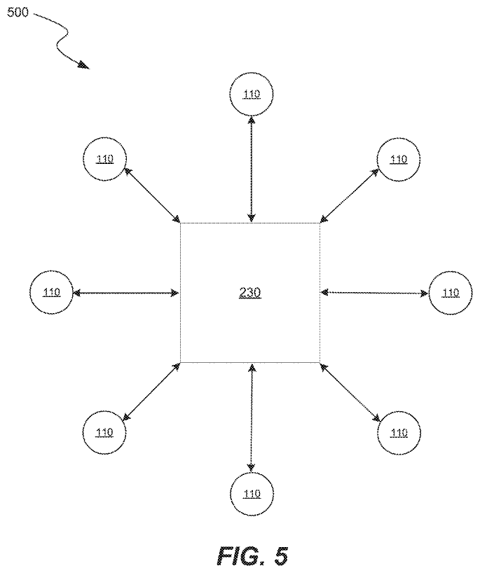

In some embodiments, one or more of the stimulus pods 110 can communicate with a control station to, for example, coordinate the delivery of stimulation to a patient at one or multiple locations. FIG. 5, for example, is a partially schematic view of a stimulus delivery system 500 configured in accordance with an embodiment of the present technology. In the illustrated embodiment, the stimulus delivery system 500 includes one or more of the stimulus pods 110 communicatively coupled to a control station 230 (shown schematically in FIG. 5). The stimulus pods 110 can communicate with the control station 230 through any accepted wireless or wired protocol, including radio frequency (RF), infrared light, laser light, visible light, acoustic energy, BLUETOOTH, WIFI, or other communication systems. Additionally, the signals can be sent and received through the patient's skin. In addition to providing a communication path among the stimulus pods 110, sending and receiving signals through the patient's skin may be particularly well suited for determining a distance between the stimulus pods 110.

The control station 230 can be a desktop or laptop computer, a smartphone, a tablet, or other device. In some embodiments, the control station 230 can be included with or integrated into a charging station, and/or can share components such as a power source, circuitry, etc., with a charging station. The control station 230 can instruct one or more of the stimulus pods 110 to apply heat, electric stimuli, vibration, or other stimulus or combination of stimuli in various patterns to the patient's body. In other embodiments, the pods 110 include a button or series of buttons through which the pods 110 can be manually operated. The possible applications are many, and include various combinations of ramp up operations, maximum intensity operations (e.g., maximum temperature or maximum electrical current, etc.), ramp down operations, stimulus soak operations, and lockout period operations (e.g., as described in detail below with reference to FIGS. 11A-18). In some embodiments, stimulus can be applied from different stimulus pods 110 at different levels and/or in different patterns. For example, a patient may place one of the stimulus pods 110 at their upper back, their lower back, and near each of their shoulders or in a different arrangement. The control station 230 can vary the stimulus application at the various zones according to a predetermined pattern. If a smartphone or other device having a screen is used as the control station 230, the screen may display a graphical representation of patient's body with indication as to where to locate the pods 110 in a particular application. Furthermore, the screen may display countdown time information for all or some of the stimulus pods 110, and/or a battery status of the stimulus pods 110.

In several embodiments, the control station 230 can detect or receive information regarding the location of the stimulus pods 110 on the patient's body, and can vary the stimulus pattern accordingly. In one embodiment, the stimulus pods 110 can be built with certain body positions in mind. In some embodiments, the stimulus pods 110 can carry body position labels to instruct the patient to apply the stimulus pods 110 according to the label. For example, in a set of four stimulus pods, two can be marked “shoulders,” a third can be marked “lower back,” and a fourth can be marked “upper back.” In some embodiments, the anchors 120 can communicate their location to the stimulus pod 110. For example, the anchors 120 can include passive identifiers such as RFID tags or other simple, passive devices for communicating with the stimulus pods 110 and/or the control station 230. In such embodiments, the anchors 120 can remain in place even when different stimulus pods 110 are swapped in and out of the anchors 120. Therefore, the stationary anchors 120 can accurately provide location information to the control station 230 independent of which specific ones of the stimulus pods 110 occupy the anchors 120.

In other embodiments, the patient can inform the control station 230 where the stimulus pods 110 are situated, and with this information the control station 230 can apply the desired stimulus pattern to the stimulus pods 110. For example, the stimulus pods 110 can fire sequentially, and the patient can indicate the location of the stimulus on a user interface. Through the user interface, the patient can also operate the system 100 and apply treatment. In some embodiments, the control station 230 can graphically display a depiction of the patient's body, and the patient can indicate to the control station 230 where the stimulus pods 110 are located on their body. Alternatively, the patient can directly control the stimulus application through the stimulus pods 110 by moving a pointing device along the graphical depiction of their body to create a virtual stimulus-massage that the patient, or a healthcare professional, controls directly. In some embodiments, the control station 230 can include a touch screen that the patient can touch to apply heat or other stimulus to various portions of their body (or to the body of another patient).

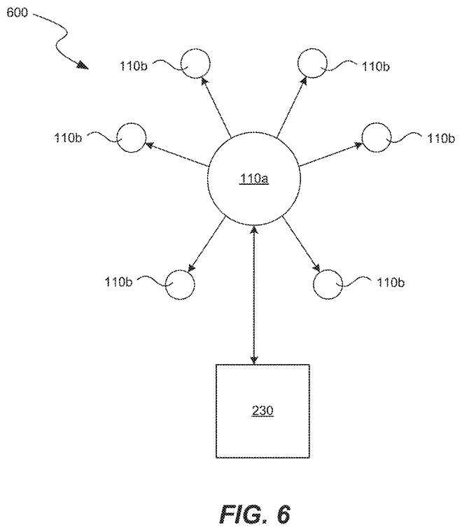

FIG. 6 is a partially schematic view of a stimulus delivery system 600 configured in accordance with another embodiment of the present technology. In the illustrated embodiment, the stimulus delivery system 600 includes a plurality of the stimulus pods 110 communicatively coupled to the control station 230 (shown schematically in FIG. 6). At least one of the stimulus pods 110 can be configured as an index pod 110a, and the other ones of the stimulus pods 110 can be configured as dummy pods 110b. In some embodiments, the relationship between the index pod 110a and the dummy pods 110b can be similar to a master/drone relationship. For example, the index pod 110a can include more sophisticated telemetry equipment than the dummy pods 110b, and can act as an intermediary between the dummy pods 110b and the control station 230. The index pod 110a may include stimulus components, such as a heating surface or vibration equipment, and can deliver stimulus just like the dummy pods 110b. Alternately, the index pod 110a can be a dedicated index pod 110a with communication equipment, but without stimulus equipment.

In some embodiments, the index pod 110a and the control station 230 can discern when two or more of the stimulus pods 110 (e.g., dummy pods 110b or index pods 110a) are near enough to one another that they can work in aggregate. If the control station 230 knows where the stimulus pods 110 are placed on the patient's body, the control station 230, through the index pod 110a, can vary the threshold distance between the stimulus pods 110 as a function of nerve density at different locations on the body. For example, if the control station 230 discerns that two or more of the stimulus pods 110 are three inches apart and on the lower back, the control station 230 can operate those ones of the stimulus pods 110 together to effectively cover the area between the stimulus pods 110 as well as the area directly contacting the stimulus pods 110. By comparison, if two or more of the stimulus pods 110 are three inches apart, but are placed on a more sensitive area, such as the patient's face or neck, the control station 230 can determine that the aggregate effect may not be perceived to reach the area between those ones of the stimulus pods 110 because of the greater nerve density. This information can be used when applying a treatment plan that calls for stimulus on a prescribed area. In some embodiments, the control station 230 can determine whether one of the stimulus pods 110 is on or near the prescribed area, and if not, whether the aggregate effect from two or more of the stimulus pods 110 can be used to carry out the treatment plan, and can execute the plan through the stimulus pods 110.

### III. Selected Embodiments of Stimulus Pod Charging Stations

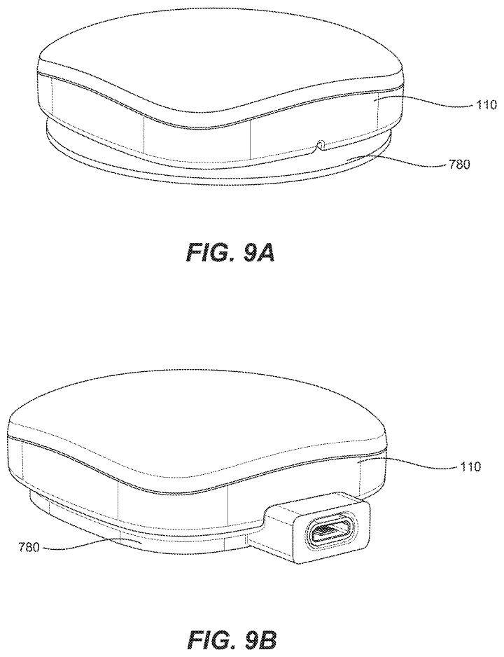

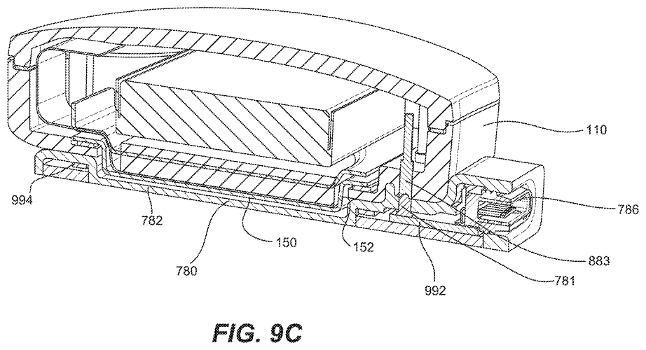

FIG. 7A is a perspective view of a charging station 780 configured in accordance with an embodiment of the present technology. FIG. 7B is an enlarged view of a portion of the charging station 780 shown in FIG. 7A. Referring to FIGS. 7A and 7B together, the charging station 780 includes a body 782 having an opening or socket 784 formed therein. As described in detail below with reference to FIGS. 9A-9C, the socket 784 is configured to receive and secure a portion of the stimulus pod 110 (e.g., the plug 152). In the illustrated embodiment, the charging station 780 also includes an electrical plug 786 configured to be coupled to a power source (e.g., an AC power source) via a power cord (not pictured). In some embodiments, the electrical plug 786 is configured to be coupled to a standard, consumer USB battery charger as is known in the art. Such USB battery chargers are low-cost solutions for recharging batteries on a broad range of devices like mobile phones, portable speakers, electronic tablets, and a host of other battery-powered devices.

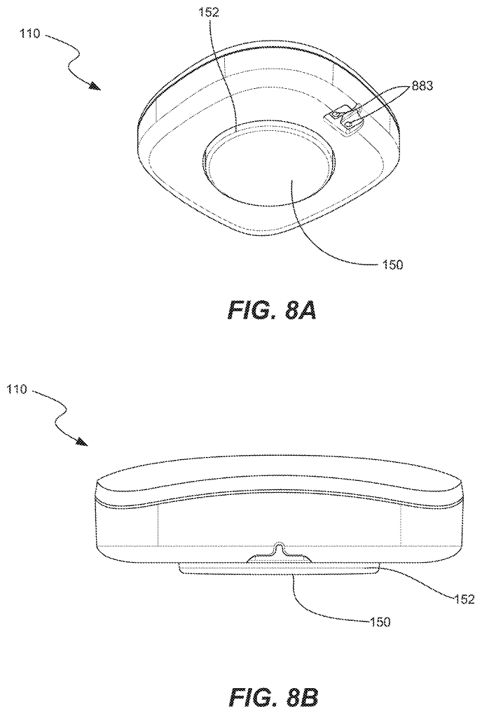

The charging station 780 can further include a pair of contacts or pins 781 configured to engage and electrically contact corresponding contacts on the stimulus pod 110 for transmitting power and/or communication signals to/from the stimulus pod 110. For example, FIGS. 8A and 8B are a bottom-perspective and a side view, respectively, of the stimulus pod 110 and showing additional features of the stimulus pod 110 in accordance with embodiments of the present technology. Referring to FIGS. 7A-8B together, the stimulus pod 110 can include contacts or pins 883 for engaging the pins 781 of the charging station 780. In some embodiments, the pins 781, 883 can be positioned in corresponding grooves, recesses, or other features to ensure proper alignment of the stimulus pod 110 and the charging station 780 such that the pins 781, 883 electrically contact one another when the stimulus pod 110 is positioned on the charging station 780.

FIGS. 9A-9C are a front-perspective view, a rear-perspective view, and a side cross-sectional view, respectively, of the stimulus pod 110 of FIGS. 8A and 8B positioned on the charging station 780 of FIGS. 7A and 7B in accordance with embodiments of the present technology. Referring to FIG. 9C, the charging station 780 can include a circuit board 992 (e.g., a printed circuit board) and a magnetic or non-magnetic metal ring 994. The metal ring 994 can engage a corresponding magnetic element in the stimulus pod 110 to secure/retain the stimulus pod 110 on the charging station 780. In the illustrated embodiment, the pins 781 and the electrical plug 786 can be electrically coupled to the circuit board 992. The circuit board 992 can be configured to transmit power to the stimulus pod 110 when the stimulus pod 110 is positioned on the charging station 780. For example, the circuit board 992 can instruct a charging coil and/or other circuitry to transmit power to a corresponding charging coil (e.g., the charging coil 165) and/or other circuitry in the stimulus pod 110. In some embodiments, the circuit board 992 can also be configured to transmit communication signals (e.g., status signals, charge levels, etc.) between the stimulus pod 110 and the charging station 780 and/or other external devices (e.g., devices coupled to the electrical plug 786). Several details of the electrical arrangement of the charging station 780 and the stimulus pod 110, such as wires and other electrical connectors, have not been shown to avoid obscuring features of the present technology.

Referring to FIGS. 7A-9C together, when the stimulus pod 110 is positioned on the charging station 780, at least a portion of the plug 152 of the stimulus pod 110 is positioned within the socket 784 of the charging station 780 such that the stimulus surface 150 is not exposed to or accessible by the patient. That is, the stimulus surface 150 can be entirely enclosed within/by the body 782 of the charging station 780 and thus cannot be attached to the patient or otherwise contact the patient during charging.

Notably, because the (e.g., mating) arrangement of the charging station 780 and the stimulus pod 110 prevents the stimulus pod 110 from being applied to the patient's body during charging, the present technology can protect the patient against the dangers of leakage currents and/or the transmission of high currents resulting from, for example, lightning strikes. Leakage current is ubiquitous within electrical and electronic systems and can be defined as the flow of current from a system's conductors to ground, either (i) directly via a properly grounded conductor, or (ii) through direct or indirect coupling to other elements of a system—for example, a human body. For supplies connected to AC power mains like battery rechargers, sources of leakage current can include capacitive couplings from electromagnetic interference (EMI) filters and from the primary to the secondary winding—or even to nearby circuits from the power transformer.

A leakage current of only 30 mA can cause breathing difficulty and ventricular fibrillation for healthy humans. Accordingly, various means of protection for protecting users of electronic devices from leakage currents and electric shocks can be built into electronic devices. These means of protection include, for example, ground fault circuit interrupter (GFCI) fail-safe protectors, insulators, air gaps, a defined ‘creepage’ (e.g., the shortest distance between two conductive paths, or a conductive path and chassis/enclosure), high-impedance isolation barriers between an electrical input and the output, etc. More generally, means of protection have been developed for various categories of products based on their electrical characteristics and the level of risk they present to users. The most stringent standards are often applied to medical technologies because of their inherent proximity to direct human contact through sensors and probes. Specifically, the International Electrotechnical Commission (IEC) technical standard 60601 covers a range of medical device safety requirements including the prevention of leakage currents. To comply with the IEC 60601 standards, the design and manufacturing of battery recharging systems requires robust protection against risks like lightning strikes translating through AC lines to users.

Consumer healthcare devices—such as the stimulus systems of the present technology—are products that fall between consumer products and medical devices, and that often rely on rechargeable battery power. Consumer healthcare devices frequently must comply with the IEC 60601 standards because of their intended direct and prolonged contact with humans. As noted above, consumer USB battery chargers are low-cost solutions for recharging batteries on a broad range of devices like mobile phones, portable speakers, electronic tablets, and other battery-powered devices. However, consumer USB battery chargers are not required to meet the requirements of the IEC 60601 standards because they are not intended for attachment to the body. Accordingly, many consumer healthcare devices cannot be compatible with USB battery chargers or else the devices would not comply with the IEC 60601 standards (and, e.g., cannot be cleared by the Food and Drug Administration (FDA)) —and thus require more complicated and relatively high cost battery charging systems.

As described in detail above, the present technology advantageously prevents a wearable device (e.g., the stimulus pod 110) from being attached to the body of a patient while it is being recharged. This eliminates the risks associated with AC line power leakage and shocks, and thus permits the utilization of lower cost consumer battery recharging systems (e.g., by eliminating the requirement that the battery recharging station comply with the IEC 60601 standards). The cost savings associated with being able to use lower cost consumer battery recharging systems (e.g., USB chargers) can be significant. For example, it is expected that the present technology will decrease the burden of factory cost by $10 on a device's battery charging system, which can translate to a retail pricing reduction of $40. Such cost savings can permit a larger segment of the population to access such wearable medical devices—even where healthcare plans may not reimburse their purchase.

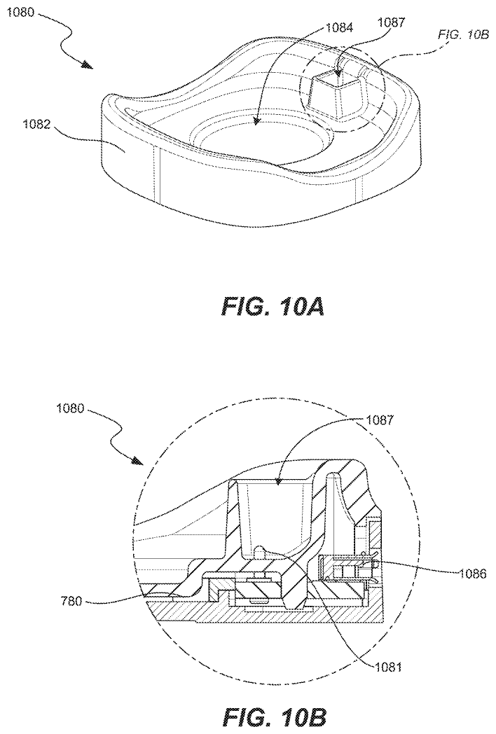

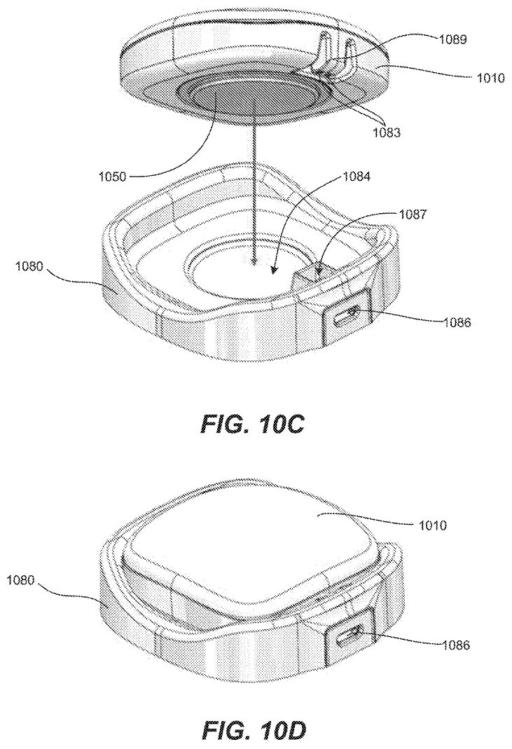

In other embodiments, the present technology can include other arrangements/configurations of a wearable device and charging station that prevent the wearable device from being worn and/or contacting a user during charging. FIG. 10A, for example, is a perspective view of a charging station 1080 configured in accordance with another embodiment of the present technology. FIG. 10B is an enlarged, side cross-sectional view of a portion of the charging station 1080 shown in FIG. 10A. FIGS. 10C and 10D are perspective views of a stimulus pod 1010 positioned above and positioned on, respectively, the charging station 1080 in accordance with embodiments of the present technology.

The stimulus pod 1010 and charging station 1080 can include some features generally similar to the features to the stimulus pod 110 and charging station 780 described in detail above. For example, referring to FIGS. 10A-10D together, the charging station 1080 includes a body 1082 having an opening or socket 1084 formed therein and configured to receive and secure a portion of the stimulus pod 1010 (e.g., to cover/enclose a stimulus surface 1050). The charging station 1080 can also include an electrical plug 1086 configured to be coupled to a power source (e.g., an AC power source) via a power cord (not pictured; e.g., a USB cord), and one or more contacts or pins 1081 configured to engage and electrically contact corresponding contacts or pins 1083 on the stimulus pod 1010 for transmitting power to the stimulus pod 1010.

In the illustrated embodiment, the pins 1081 are positioned within a recess 1087 formed in the body 1082. The stimulus pod 1010 (or another wearable device) can have a corresponding protrusion 1089 on which the pins 1083 are positioned for electrically contacting the pins 1081 when the stimulus pod 1010 is positioned on (e.g., mounted on) the charging station 1080. More particularly, the protrusion 1089 can be configured to extend into the recess 1087 such that the pins 1081, 1083 electrically contact one another. The recess 1087 can be specifically configured (e.g., sized and shaped) to prevent or at least substantially inhibit a user from (i) directly contacting the pins 1081 with their fingertip (e.g., an artificial fingertip as defined by the IEC 60601 standards) and/or (ii) indirectly receiving an electrical shock during a lightning strike while the charging station 1080 is coupled to an AC power source. In the illustrated embodiment, for example, the recess 1087 is sized to provide at least 5 mm of air clearance with respect to a 12 mm (artificial) fingertip, and to provide at least 8 mm of surface separation distance (i.e., creepage). Accordingly, in some embodiments the charging station 1080 is specifically configured to meet the requirements of the IEC 60601 standards.

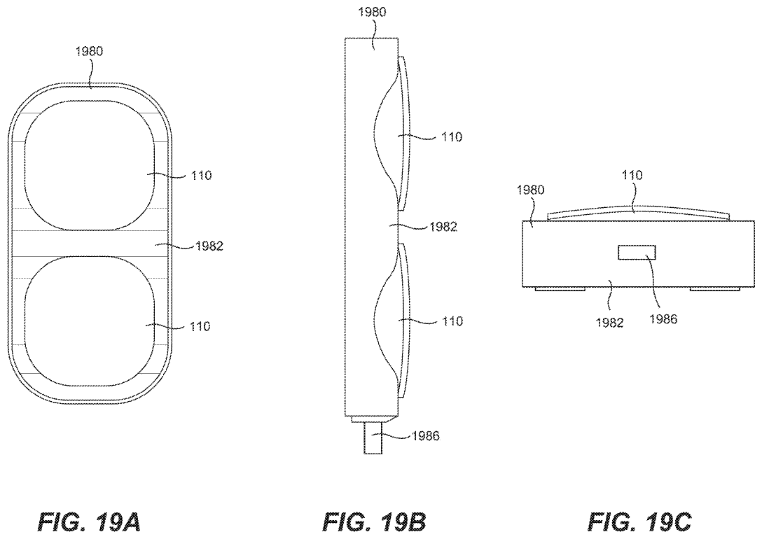

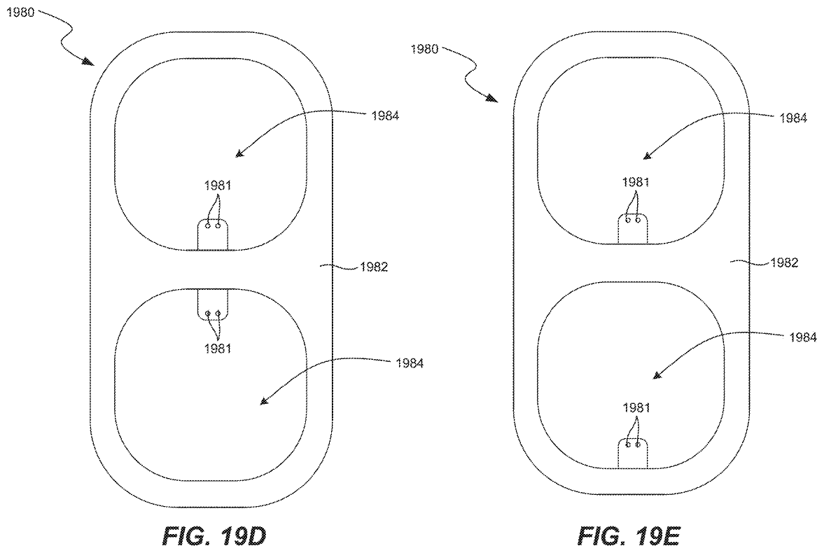

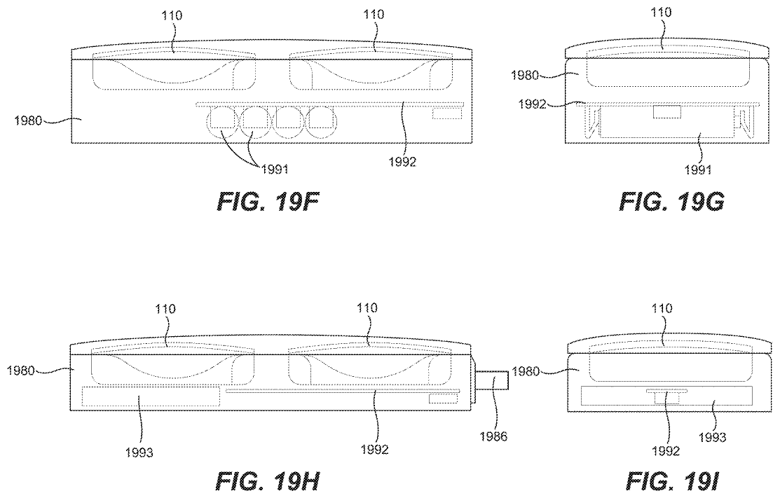

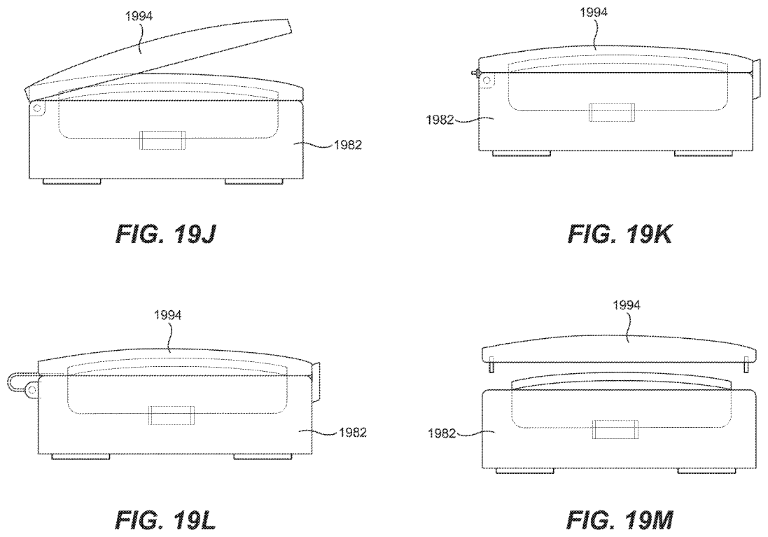

FIGS. 19A-19C are a top view, a side view, and end view, respectively, of a charging station 1980 having the stimulus pods 110 positioned thereon in accordance with another embodiment of the present technology. FIGS. 19E and 19F are top views of the charging station 1980 with the stimulus pods 110 removed. Referring to FIGS. 19A-19E together, the charging station 1980 includes a body 1982 having more than one (e.g., two) openings or sockets 1984 formed therein and that are each configured to receive and secure a portion of one of the stimulus pods 110 (e.g., to cover/enclose the stimulus surface 150). In other embodiments, the charging station 1980 can include more than the two illustrated sockets 1984 such that the charging station 1980 can charge more than two of the stimulus pods 110 simultaneously. In some embodiments, the charging station 1980 can include an electrical plug 1986 configured to be coupled to a power source (e.g., an AC power source) via a power cord (e.g., a USB cord), and one or more contacts or pins 1981 positioned in the sockets 1984 and configured to engage and electrically contact corresponding contacts or pins of the stimulus pods 110. As shown in FIGS. 19D and 19E, the pins 1981 can be positioned at various locations/positions within the sockets 1984.

In some embodiments, the charging station 1980 can be configured as a portable, cordless station that includes one or more batteries. For example, FIGS. 19F and 19G are side and end cross-sectional views, respectively, of the charging station 1980 having the stimulus pods 110 positioned thereon in accordance with an embodiment of the present technology. Referring to FIGS. 19F and 19G together, the charging station 1980 can include one or more replaceable batteries 1991 that are electrically coupled to a circuit board 1982. In some embodiments, the batteries 1991 can be rechargeable batteries while, in other embodiments the batteries 1991 can be AA, AAA, or other types of disposable batteries. FIGS. 19H and 19I are side and end cross-sectional views, respectively, of the charging station 1980 having the stimulus pods 110 positioned thereon in accordance with another embodiment of the present technology. Referring to FIGS. 19H and 19I together, in the illustrated embodiment the charging station 1980 includes a battery 1993 that can be (e.g. permanently) secured within the body 1982 of the charging station 1980 and that is configured to be recharged via, for example, the electrical plug 1986. In some embodiments, the battery 1993 can a lithium ion battery or other battery having a flat profile that enables the charging station 1980 to be made relatively thinner (e.g., as compared to the embodiment illustrated in FIGS. 19F and 19G).

In some embodiments, the charging station 1980 can include a lid 1994 that can be removably or permanently coupled to the body 1982 for enclosing the stimulus pods 110 during charging and/or protecting the sockets 1984 from debris or contamination. For example, FIGS. 19J-19M are end views of charging station 1980 having a lid 1994 configured in accordance with embodiments of the present technology. In the embodiment illustrated in FIG. 19J, the body 1982 includes an integrated hinge having a retaining pin (e.g., a metal or plastic pin) that permits the lid 1994 to pivot relative to the body 1982. In the embodiment illustrated in FIG. 19K, the body 1982 includes a molded-in living hinge and a latch. In the embodiment illustrated in FIG. 19L, the charging station includes a separate elastomeric hinge and a latch. In the embodiment illustrated in FIG. 19M, the lid 1994 is configured to snap on/off the body 1982. In other embodiments, the lid 1994 can be secured to the body 1982 via other suitable means.

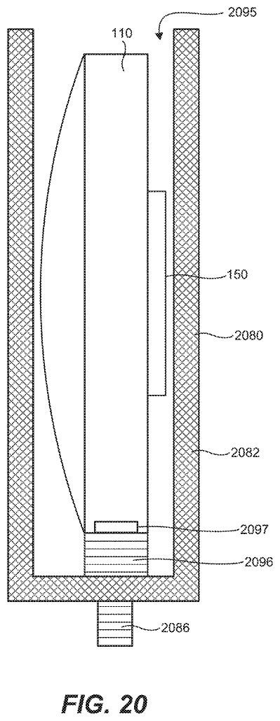

FIG. 20 is a side view of a charging station 2080 having the stimulus pod 110 positioned therein in accordance with another embodiment of the present technology. In the illustrated embodiment, the charging station 2080 includes a body 2082 that defines an enclosure 2095 configured to receive the stimulus pod 110 fully or partially therein. The charging station 2080 can further include an electrical connector 2096 that mates with an electrical connector 2097 of the stimulus pod 110 when the stimulus pod is positioned within the enclosure 2095. The charging station 2080 can further include an electrical plug 2086 configured to be coupled to a power source (e.g., an AC power source) via a power cord (e.g., a USB cord) for charging the stimulus pod 110. Notably, the stimulus surface 150 of the stimulus pod 110 is inaccessible to the user when the stimulus pod 110 is positioned in the enclosure 2095 of the charging station 2080 during charging.

From the foregoing, it will be appreciated that specific embodiments have been described herein for purposes of illustration, but that various modifications may be made without deviating from the spirit and scope of the present technology. For example, in particular embodiments, details of the disclosed charging stations and/or stimulus pods or other wearable devices may be different than those shown in the foregoing Figures. For example, a charging station and a stimulus pod or other wearable device according to the present technology can have any suitable arrangement for preventing a user from wearing or contacting a stimulus surface of the pod during charging. In some embodiments, for example, a stimulus pod can have a power plug incorporated into a stimulus surface that contacts the patient during use. In such embodiments, the position and orientation of a charging cord (e.g., a USB cord) can interfere with or entirely prevent the user from wearing the stimulus pod during use.

### IV. Selected Embodiments of Methods of Applying Stimuli

The stimulus delivery systems of the present technology can be used to apply therapeutic stimuli to a patient in many different patterns, magnitudes, cycles, etc. For example, a control unit (e.g., the control station 230) can be used to activate and control one or more wearable devices (e.g., the stimulus pods 110) to apply stimuli according to a predetermined heating cycle and/or pattern. In some embodiments, the stimulus pods 110 are configured to be placed in various locations on the skin of the patient to provide therapeutic heat treatment for relieving pain. The following disclosure details a few specific methods of applying stimuli using the delivery systems of the present technology. However, one skilled in the art will appreciate that the present technology can be used in many different manners to alleviate pain, treat ailments, etc., without deviating from the scope of the present technology. Moreover, while reference is made herein to the stimulus pod system 100, one skilled in the art will appreciate that the following methods can be carried out using other suitable devices—for example, those described in detail in U.S. Pat. No. 7,871,427, tilted “APPARATUS AND METHOD FOR USING A PORTABLE THERMAL DEVICE TO REDUCE ACCOMMODATION OF NERVE RECEPTORS,” and filed Feb. 8, 2006; and U.S. Pat. No. 8,579,953, titled “DEVICES AND METHODS FOR THERAPEUTIC HEAT TREATMENT,” and filed Dec. 8, 2008, each of which incorporated herein by reference in its entirety.

### 1. Selected Embodiments of Low-Level Heating and Cooling Combined with Intermittent High-Level Heating

In some embodiments, the present technology can be configured to apply a continuous amount of low-level heat combined with discrete amounts or intermittent bursts of high-level heat to a patient. As described below, the bursts of heat can be at distinct locations within or around the areas producing the low-level heat. The low-level heat can be maintained as a constant application of heat while the high-level heat is applied in intermittent bursts (e.g., milliseconds in some embodiments).

To better appreciate the benefits of the combination of the continuous low temperature heat and the intermittent high temperature heat, it is helpful to understand the body's reaction to heat. The human body is generally sensitive to heat, with certain body parts having a higher sensitivity than other body parts. The body's sensitivity to heat is recognized by thermal receptors located in the skin and subcutaneous tissue. FIG. 11A illustrates a mapping of the thermal receptors 1102 of a human leg 1104 and foot 1106. As shown in FIG. 11A, the receptors 1102 have defined receptive fields with little overlap between the fields. The receptors 1102 are excited by heat that is applied to the skin. When the receptors 1102 become excited from the applied heat, they send signals to stimulate the brain. The brain can accordingly coordinate other bodily functions in response to the signals sent from the receptors 1102. For example, the brain can signal to the body to produce endorphins as an analgesic response to the applied heat.

The thermal receptors located throughout the body can be excited or activated at different temperatures. FIG. 11B, for example, is a graph 1108 of the excitation of various receptors versus applied heat. The x-axis of FIG. 11B represents mechanical pressure in mN of excited receptors, and the y-axis represents the temperature in degrees C. of the applied heat. As illustrated by the graph 1108, the majority of the excitation of the thermal receptors occurs at temperatures above 42° C., although some excitation does occur at temperatures below 42° C. The excitation also generally peaks below 50° C. Accordingly, in certain embodiments of the present disclosure, bursts of heat in the range of 42-55° C. are applied to discrete areas of skin to excite the receptors. The thermal bursts may be applied in combination with low level heating (e.g., heating below 42° C.). In other embodiments, however, the thermal bursts can include temperatures higher or lower than the range of 42-55° C. For the purposes of this disclosure, thermal bursts can be defined as the application of increased heat in discrete areas where the temperature of the burst ranges from 0.1° C. to 25° C. or more above the baseline temperature of the continuous low-level heat application. The thermal bursts can include a ramp up speed ranging from milliseconds to minutes to reach a maximum temperature. In addition, and as described below, the size of the area applying the thermal burst is generally relatively small in comparison to the area applying the low-level heat.

In some embodiments, a method of applying heat to a living body includes applying a constant amount of heat to a first defined region of the body at a first temperature (e.g., via a first one of the stimulus pods 110). The method can also include applying intermittent amounts of heat to a second defined region of the body (e.g., via a second one of the stimulus pods 110). The intermittent amounts of heat may be applied at a second temperature greater than the first temperature. According to further embodiments, the second region overlaps the first region. According to still further embodiments, the intermittent amounts of heat are delivered at pre-selected, focused points wherein the surface area of the second region is smaller than the surface area of the first region.

A method configured in accordance with another embodiment of the disclosure includes a method of exciting thermal receptors in a living organism. The method includes heating a first portion of skin with a generally constant amount of heat at a baseline temperature (e.g., via a first one of the stimulus pods 110), and heating a second portion of skin with a burst of heat at a temperature above the baseline temperature (e.g., via second one of the stimulus pods 110) while heating the first portion of skin with the generally constant amount of heat.

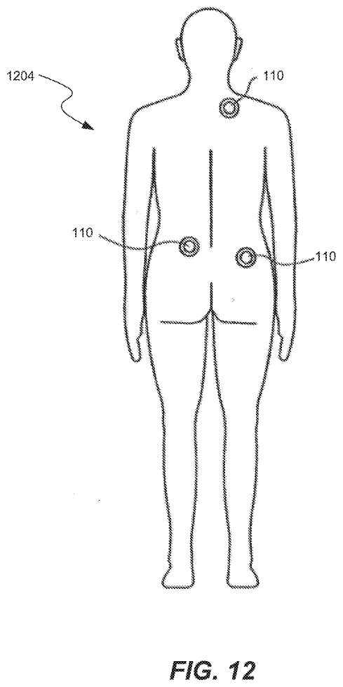

FIG. 12 is a back view of a human form 1204 wearing a plurality of the stimulus pods 110 (e.g., at the shoulder, lower back, and hip). The stimulus pods 110 can be configured to provide continuous low-level heating with periodic bursts or impulses of high-level heat, and can be applied simultaneously to various areas of the body 1204 and can be used in conjunction with one another or independently to provide pain relief. Therefore, the stimulus pods 110 can accommodate users who suffer from pain in areas located in more than one region of the body thus requiring simultaneous treatment. For example, the treatment of conditions such as fibromyalgia, dysmenorrhea, PMS, back and neck pain, sports related injuries, etc., may greatly benefit from locating the stimulus pods 110 at different positions to simultaneously treat one or more painful areas.

The combination of the continuous low-level heating and intermittent high-level heating at discrete, focused regions provides several advantages over conventional heating systems. The augmentation of the continuous heating (or cooling), for example, provides enhanced pain relief by promoting blood flow, increasing flexibility, and relaxing muscles, ligaments, and other tissues. The illustrated configuration achieves enhanced pain relief by providing a strong stimulation of the thermal receptors in the skin and subcutaneous tissues of the body by rapidly changing temperatures. The variations of the temperatures from the thermal bursts reduce or eliminate the accommodation of the receptors to the stimuli. For example, when heat is applied to the body at a constant temperature, the receptors can accommodate the constant heat thus reducing the stimulation. The intermittent bursts of heat, however, can at least partially prevent the receptors from adjusting to the heat by not providing sufficient time for accommodation. This is especially effective when the intermittent bursts of heat are provided by stimulus pods of a relatively small surface area, for example 2″ by 2″, or more particularly 1″ by 1″, or even more particularly, ½″ by ½″. This is unlike conventional heating systems that do not provide the ability to disrupt the accommodation of the receptors. Accordingly, the intermittent focused bursts of heat, combined with the constant heat, provide for better receptor stimulation resulting in better analgesic results.

### 2. Selected Embodiments of Heat Cycling

In some embodiments, the present technology can be used to provide heat to a patient to reduce accommodation of thermal nerve receptors of a subject. The method includes increasing the temperature of a heating element (e.g., one or more of the stimulus surfaces 150 of the stimulus pods 110) to provide a first temperature ramp-up period, holding the temperature of the heating element at a predetermined therapeutic level, decreasing the temperature of the heating device during a ramp-down period, and holding the temperature of the heating device at a predetermined soak level, wherein the soak level temperature is above a basal temperature, and wherein the soak level temperature is less than the therapeutic level temperature by at least 1° C.

In operation, the heating device (e.g., one or more of the stimulus pods 110) may deliver heat intermittently. The heat may be applied for a period long enough to heat the skin to a desired level; upon reaching the desired skin temperature the device turns off and the skin is allowed to cool; after a preprogrammed interval the device may reactivate the heat unit and the cycle repeats. Alternatively, multiple cycles may be delivered sequentially for a predetermined duration.

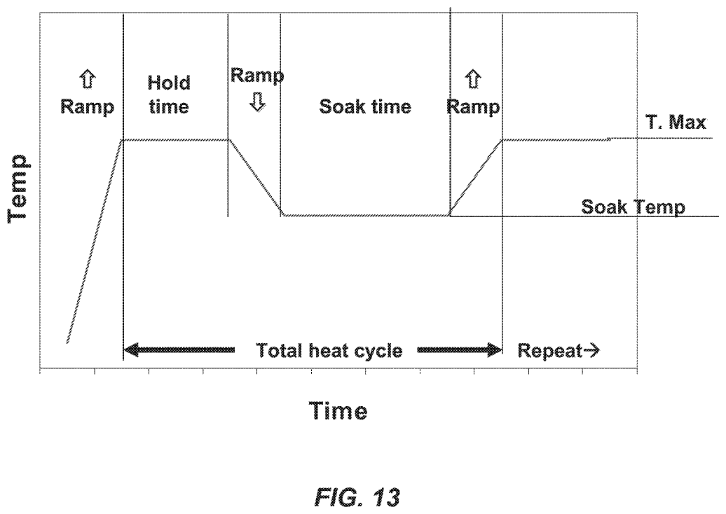

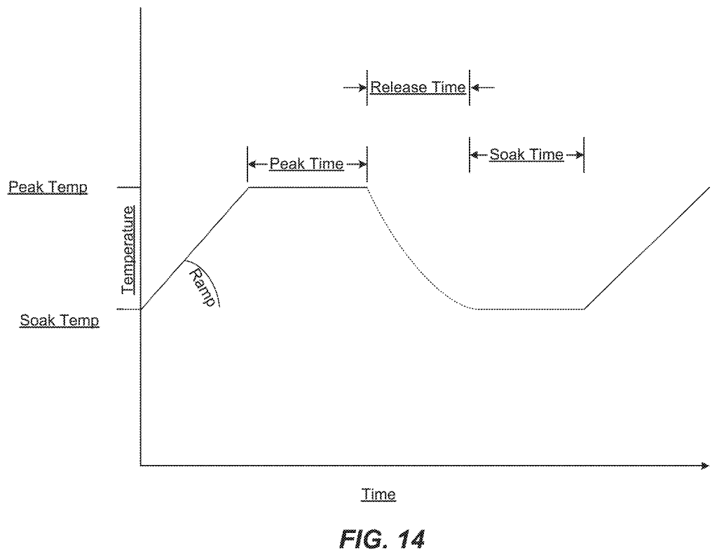

FIG. 13, for example, is a graph of temperature versus time illustrating a variable heat cycle for a heating element configured in accordance with an embodiment of the present technology. In the illustrated embodiment, the variable heat cycle includes a therapeutic temperature hold phase, a ramp-down phase, a soak phase, and a ramp-up phase. FIG. 14 is a graph of temperature versus time illustrating a variable heat cycle for the heating element configured in accordance with another embodiment of the present technology. In the illustrated embodiment, the variable heat cycle includes a ramp-up phase, a peak-time hold phase, a release phase, and a soak phase. The variable heat cycles shown in FIGS. 13 and 14 provides many advantages to the user. One advantage is an increased effectiveness of the thermal stimulation because the variable heat cycles prevent the nervous system receptors from accommodating to the thermal stimulation. For example, when a steady state heat is delivered, over time the thermal nerve receptors accommodate the thermal stimulation and emit a reduced response, thus reducing or eliminating the therapeutic effect of the thermal stimulation. When the variable heat cycle of the current embodiment is delivered, the thermal nerve receptors are not given time to accommodate to the thermal stimulation before the thermal stimulation is reduced, and therefore, the nerve receptors are reactivated with each variable heat cycle.

Without being bound by theory, the present technology provides thermal stimulation to the skin of a user; the thermal stimulation provides pain relief to the nervous system by stimulating the nervous system, but not allowing the thermal nerve receptors time to accommodate to the stimulation. In general, the nervous system is continuously attempting to accommodate to stimulants. When presented with a stimulant, the nervous system will react to the stimulant with a nerve response. Over time, the nervous system accommodates to the stimulation and provides a lesser response to the stimulation. However, if the stimulation is applied and then removed or reduced to allow the nervous system to reset or return to a baseline response mode, the thermal nerve receptors are not given the opportunity to accommodate to the stimulation and thus react anew to each introduction of the stimulation.

Another advantage of the variable heat cycles of the present technology is that multiple therapeutic methodologies are applied in one cycle, namely, inhibiting nociception and increasing blood flow. The direct thermal stimulation in the peak time or therapeutic temperature hold provides direct stimulation of the nerves through heat and thus provides a counter-irritant to pain. Additionally, the soak phase is held at a temperature higher than the basal body temperature of the user, thus allowing continued therapeutic effects by improving the blood flow to the region and providing muscle relaxation while allowing the thermal nerve receptors to return to a baseline response mode. Yet another advantage of the variable heat cycles is reduced power demand and consumption during the ramp-down or release phase when the thermal device does not draw power from the power supply, or draws reduced power from the power supply. Reduced power consumption results in a more efficient device with a longer life cycle and provides cost savings.

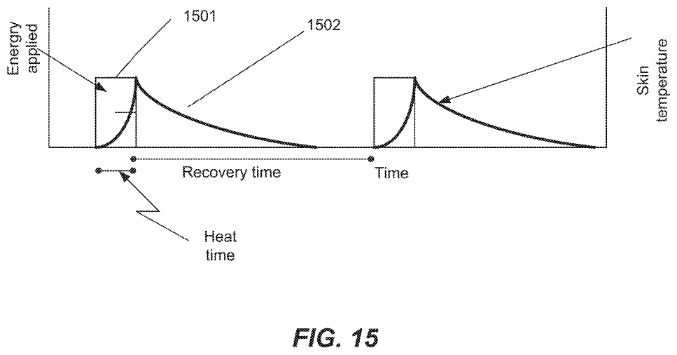

FIG. 15 is a graph of energy applied versus time illustrating the energy applied to the system and the resultant skin temperature of a patient in accordance with an embodiment of the present technology. In FIG. 15, bars 1501 indicate how long and how much energy is applied via the stimulus pods 110. The heat applied is measured on an arbitrary scale on the left, and lines 1502 indicate estimated skin temperature for each profile and the skin temperature is indicated on an arbitrary scale on the right.

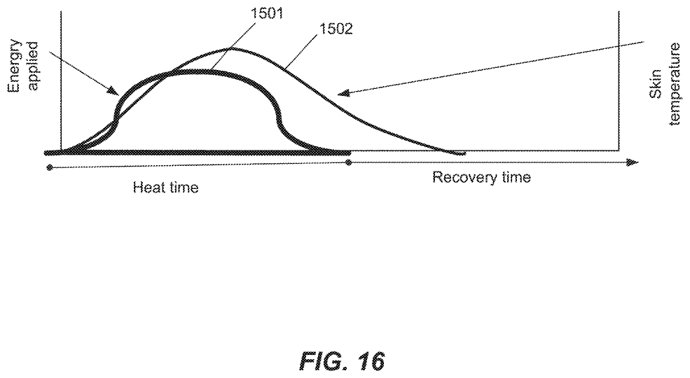

FIG. 16 is a graph of energy applied versus time illustrating a sine wave energy applied pattern and the resultant skin temperature of a patient in accordance with an embodiment of the present technology. In other embodiments, the pattern of applied energy can be a square, crescendo, de-crescendo, intermittent, or any other conceivable pattern. Thus, there are at least four variables that can be adjusted to ensure optimal analgesia; duration of heating “heat time,” “recovery times” between heat times, intensity of heating, and pattern of heating (sine wave (as shown in FIG. 16), square wave, saw tooth etc).

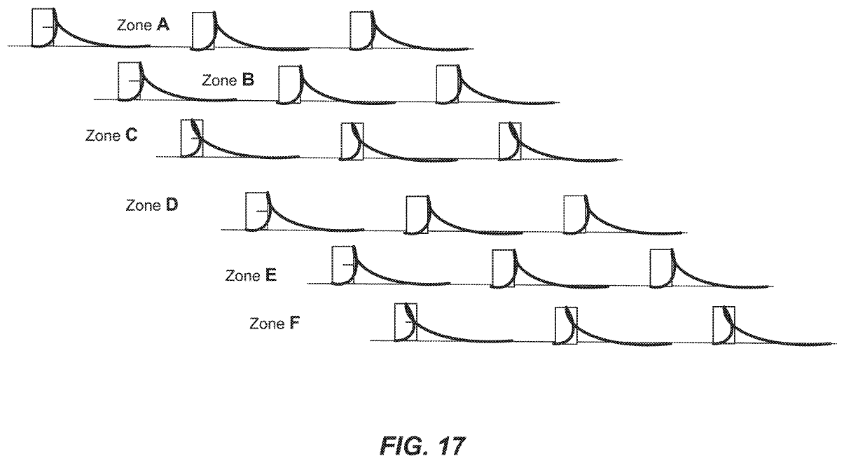

FIG. 17 illustrates energy applied to exemplary thermal zones A, B, C, D, E and the resultant skin temperature of a patient in accordance with an embodiment of the present technology. In some embodiments, the thermal zones A-E can correspond to zones under or proximate to different ones of the stimulus pods 110. In the illustrated embodiment, the skin temperature in the thermal zones A-E has a cascading pattern. In particular, heat is applied to each zone in a sequential pattern. That is, as energy is applied to zone A, while zone B rests, then zone B heats while zone A rests, and then zone C heats while zone D rests, and so forth. This has the effect of a wave of heat being passed from zone A to zone E and back again. The principle of moving heat zones can be applied vertically horizontally or both to achieve a checkerboard effect or any other conceivable pattern. In other embodiments, the system can deliver any conceivable pattern. For example, heat can be applied in a non-uniform manner. Similarly, by taking advantage of individually controllable heat regions or heat zones (e.g., corresponding to different ones of the stimulus pods 110), the heat can be applied sequentially or in any other imaginable pattern. Sequential heating of individual heat regions as drawn in FIG. 17 can enable an entirely different therapeutic sensation to be achieved as compared with heating them all at the same time.

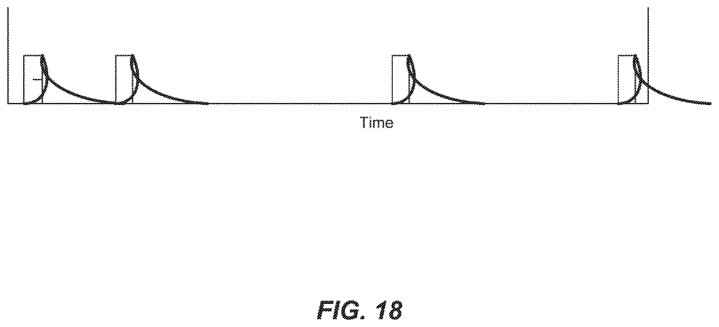

FIG. 18 illustrates an on-demand pattern of variable heat cycles over time as requested by a subject in accordance with an embodiment of the present technology. For example, the patient can press an actuator on the stimulus pod 110, such as a lever, a switch, a pressure sensor, or any other activation device as is known in the art, to demand heat on an as-needed basis. FIG. 18 shows, on an arbitrary time base, the patient demanding analgesia four times. The pattern of heat delivered by the system could be constant or preprogrammed onto a control unit.

One expected advantage of the present technology is that the heating devices are portable and can be conveniently worn by the subject such that pain relief is available as needed. According to aspects of the present technology, the heating devices are designed to relieve pain or assist with healing in a variety of medical conditions such as low, mid, or upper back pain, muscular pain, dysmenorrhea, headaches, fibromyalgia, post-herpetic neuralgia, nerve injuries and neuropathies, injuries to extremities, and sprains and strains. Another expected advantage is that greater pain relief will be realized by the user because they will be able to control the frequency and duration of the treatment. Another expected advantage is increased efficacy of TENS when used in combination with the system described herein.

### V. Further Examples

The following examples are illustrative of several embodiments of the present technology:

1. A system, comprising:
a charging station; and
a device wearable by a user,
   wherein the device is configured to be positioned on the charging station and to be electrically coupled to the charging station for receiving power, and
   wherein the charging station is configured to inhibit the wearable device from being worn by the user when the device is positioned on the charging station and receiving power.

2. The system of example 1 wherein the device includes a stimulation surface configured to be placed against the user to provide a stimulus to the user.

3. The system of example 2 wherein the device includes a plug for receiving the power, and wherein the plug extends through the stimulus surface to inhibit the device from being worn by the user.

4. The system of example 2 wherein the charging station is configured to physically surround the stimulation surface when the device is positioned on the charging station to inhibit the device from being worn by the user.

5. The system of any one of examples 1-4 wherein the charging station is configured to physically encompass the device when the device is positioned on the charging station to inhibit the device from being worn by the user.

6. The system of any one of examples 1-5 wherein the charging station is configured to be connected to a power source via a USB connector.

7. The system of any one of examples 1-5 wherein the charging station is configured to be connected to a power source via a connector that is not a USB connector.

8. The system of any one of examples 1-7 wherein the charging station includes electrical contacts configured to mate with corresponding electrical contacts of the device, and wherein the charging station is configured to prevent an artificial fingertip as defined by IEC-60601 from coming within a specific distance of the electrical contacts either (a) through the air or (b) along a surface of the charging station.

9. The system of example 8 wherein the charging station defines an air gap proximate to the electrical contacts of about 5 mm.

10. The system of example 8 or example 9 wherein the charging station defines a creepage from the electrical contacts of about 8 mm.

11. The system of any one of examples 8-10 wherein—
the device includes a protrusion having first electrical contacts;
the charging station includes a recess and second electrical contacts positioned within the recess; and
when the device is positioned on the charging station, the protrusion is configured to extend into the recess such that the first electrical contacts contact corresponding ones of the second electrical contacts for receiving the power.

12. The system of any one of examples 1-11 wherein the charging station includes one or more batteries configured to supply power to the device.

13. A device configured to charge a stimulus pod having a stimulation surface configured to be positioned adjacent the skin of a user to provide a stimulus to the user, the device comprising:
a housing having a socket shaped to receive the stimulation surface of the stimulus pod;
power circuitry positioned within the housing; and
contacts electrically coupled to the power circuitry,
   wherein the contacts are configured to electrically contact the stimulation pod to provide power to the stimulation pod when the stimulation surface is received in the socket, and
   wherein the housing inhibits the stimulation surface from contacting the user when the stimulation surface is received in the socket.

14. The device of example 13 wherein the housing further includes a recess, and wherein the contacts are positioned within the recess.

15. The device of example 14 wherein the recess defines a creepage from the contacts of about 8 mm.

16. The device of any one of examples 13-15 wherein the recess defines an air gap proximate to the contacts of about 5 mm.

17. The device of any one of examples 13-16 wherein the power circuitry is configured to be electrically coupled to an external power source via a USB connector.

18. The device of any one of examples 13-16 wherein the power circuitry includes a power source.

19. A method of charging a wearable device having a stimulation surface, the method comprising:
positioning the wearable device on a charging station such that (a) the wearable device is electrically coupled to the charging station for receiving power and (b) the stimulation surface cannot be positioned against a user while the device is receiving power.

20. The method of example 19 wherein positioning the wearable device on the charging station includes positioned the stimulation surface of the wearable device in a socket of the charging station such that the user cannot contact the stimulation surface while the device is receiving power.

### VI. Conclusion

Unless the context clearly requires otherwise, throughout the description and the claims, the words “comprise,” “comprising,” and the like are to be construed in an inclusive sense, as opposed to an exclusive or exhaustive sense; that is to say, in the sense of “including, but not limited to.” As used herein, the terms “connected,” “coupled,” or any variant thereof, means any connection or coupling, either direct or indirect, between two or more elements; the coupling of connection between the elements can be physical, logical, or a combination thereof. Additionally, the words “herein,” “above,” “below,” and words of similar import, when used in this application, shall refer to this application as a whole and not to any particular portions of this application. Where the context permits, words in the above Detailed Description using the singular or plural number may also include the plural or singular number respectively. The word “or,” in reference to a list of two or more items, covers all of the following interpretations of the word: any of the items in the list, all of the items in the list, and any combination of the items in the list.

The above detailed description of embodiments of the present technology is not intended to be exhaustive or to limit the present technology to the precise form disclosed above. While specific embodiments of, and examples for, the present technology are described above for illustrative purposes, various equivalent modifications are possible within the scope of the present technology, as those skilled in the relevant art will recognize. The teachings of the present technology provided herein can be applied to other systems, not necessarily the system described above. The elements and acts of the various embodiments described above can be combined to provide further embodiments. All of the above patents and applications and other references, including any that may be listed in accompanying filing papers, are incorporated herein by reference. Aspects of the present technology can be modified, if necessary, to employ the systems, functions, and concepts of the various references described above to provide yet further embodiments of the present technology.

These and other changes can be made to the present technology in light of the above Detailed Description. While the above description details certain embodiments of the present technology and describes the best mode contemplated, no matter how detailed the above appears in text, the present technology can be practiced in many ways. Details of the system may vary considerably in its implementation details, while still being encompassed by the present technology disclosed herein. As noted above, particular terminology used when describing certain features or aspects of the present technology should not be taken to imply that the terminology is being redefined herein to be restricted to any specific characteristics, features, or aspects of the present technology with which that terminology is associated. In general, the terms used in the following claims should not be construed to limit the present technology to the specific embodiments disclosed in the specification, unless the above Detailed Description section explicitly defines such terms. Accordingly, the actual scope of the present technology encompasses not only the disclosed embodiments, but also all equivalent ways of practicing or implementing the present technology.

I/We claim:

1. A system, comprising:
a charging station; and
a device wearable by a user,
wherein the device is configured to be positioned on the charging station and to be electrically coupled to the charging station for receiving power, and
wherein the charging station is configured to inhibit the wearable device from being worn by the user when the device is positioned on the charging station and receiving power.

2. The system of claim 1 wherein the device includes a stimulation surface configured to be placed against the user to provide a stimulus to the user.

3. The system of claim 2 wherein the device includes a plug for receiving the power, and wherein the plug extends through the stimulus surface to inhibit the device from being worn by the user.

4. The system of claim 2 wherein the charging station is configured to physically surround the stimulation surface when the device is positioned on the charging station to inhibit the device from being worn by the user.

5. The system of claim 1 wherein the charging station is configured to physically encompass the device when the device is positioned on the charging station to inhibit the device from being worn by the user.

6. The system of claim 1 wherein the charging station is configured to be connected to a power source via a USB connector.

7. The system of claim 1 wherein the charging station is configured to be connected to a power source via a connector that is not a USB connector.

8. The system of claim 1 wherein the charging station includes electrical contacts configured to mate with corresponding electrical contacts of the device, and wherein the charging station is configured to prevent an artificial fingertip as defined by IEC-60601 from coming within a specific distance of the electrical contacts either (a) through the air or (b) along a surface of the charging station.

9. The system of claim 8 wherein the charging station defines an air gap proximate to the electrical contacts of about 5 mm.

10. The system of claim 8 wherein the charging station defines a creepage from the electrical contacts of about 8 mm.

11. The system of claim 8 wherein—
the device includes a protrusion having first electrical contacts;
the charging station includes a recess and second electrical contacts positioned within the recess; and
when the device is positioned on the charging station, the protrusion is configured to extend into the recess such that the first electrical contacts contact corresponding ones of the second electrical contacts for receiving the power.

12. The system of claim 1 wherein the charging station includes one or more batteries configured to supply power to the device.

13. A device configured to charge a stimulus pod having a stimulation surface configured to be positioned adjacent the skin of a user to provide a stimulus to the user, the device comprising:
a housing having a socket shaped to receive the stimulation surface of the stimulus pod;
power circuitry positioned within the housing; and
contacts electrically coupled to the power circuitry,
wherein the contacts are configured to electrically contact the stimulation pod to provide power to the stimulation pod when the stimulation surface is received in the socket, and
wherein the housing inhibits the stimulation surface from contacting the user when the stimulation surface is received in the socket.

14. The device of claim 13 wherein the housing further includes a recess, and wherein the contacts are positioned within the recess.

15. The device of claim 14 wherein the recess defines a creepage from the contacts of about 8 mm.

16. The device of claim 14 wherein the recess defines an air gap proximate to the contacts of about 5 mm.

17. The device of claim 13 wherein the power circuitry is configured to be electrically coupled to an external power source via a USB connector.

18. The device of claim 13 wherein the power circuitry includes a power source.

19. A method of charging a wearable device having a stimulation surface, the method comprising:
positioning the wearable device on a charging station such that (a) the wearable device is electrically coupled to the charging station for receiving power and (b) the stimulation surface cannot be positioned against a user while the device is receiving power.

20. The method of claim 19 wherein positioning the wearable device on the charging station includes positioned the stimulation surface of the wearable device in a socket of the charging station such that the user cannot contact the stimulation surface while the device is receiving power.
